(19) United States
(12) Patent Application Publication
(10) Pub. No.: US 2023/0062335 A1
(43) Pub. Date: Mar. 2, 2023

(54) System and Method for Providing Drive-Through Healthcare Services

(71) Applicant: HealthBaar LLC, Grand Rapids, MI (US)

(72) Inventor: Nathan BAAR, Grand Rapids, MI (US)

(73) Assignee: HealthBaar LLC, Grand Rapids, MI (US)

(21) Appl. No.: 17/465,607

(22) Filed: Sep. 2, 2021

(51) Int. Cl. G16H 40/67; G16H 10/20; G06Q 10/10

(52) U.S. Cl. CPC G16H 40/67; G16H 10/20; G06Q 10/1095

(57) ABSTRACT

Systems and methods for providing drive-through healthcare services are provided. Individuals can drive to a convenient drive-through healthcare facility, with or without an appointment, and receive a full range of medical diagnostic and treatment services. All services are provided to the individual while she remains in her car. At the conclusion of the services, the individual drives away from the drive-through healthcare facility, and the system transmits to the individual a complete summary of all services that have been provided, including any test results, any diagnosis results, all treatments received, any prescriptions recommended, and any referrals or appointments for future treatment and follow-up.

### BACKGROUND

Persons are regularly in need of healthcare services to both maintain their health and to address injuries and ailments. In the distant past, healthcare providers, whether licensed doctors and nurses or trained medical assistants such as midwifes, would go to the location of the patient to provide healthcare services. At the patient's location, the health, injuries, or ailments of the patient would be diagnosed and treatment would be administered, if possible. Such healthcare services could be a wide scope of services addressing the health of the patient, ranging from a regular checkup to setting a broken bone, from stitching up a cut to childbirth, and from administering a tetanus shot to surgery.

In more recent years, patients typically would transport themselves to a medical facility to receive healthcare services. Such facilities could include a doctor's office, a hospital, an urgent care facility, and the like. However, such facilities may be remote from the patient and may require significant lead time for receiving an appointment to see a healthcare provider. Further, a single initial appointment with a healthcare provider may result in a subsequent appointment with that same provider or a different provider to address the particular health issue being suffered by the patient. Such sequential appointments may easily result in the patient not receiving treatment directly addressing the health issue of the patient.

What is needed is a convenient, relatively nearby facility where the patent can drive to or be transported to; and where the patent can receive the needed full-range of diagnostic and treatment healthcare services in a drive-through environment.

### SUMMARY

This summary is provided to present a selection of concepts that are further described in greater detail below in the Detailed Description. This summary is not intended to identify important or required features of the claimed subject matter, nor is it intended to establish the scope of the claimed subject matter.

According to certain embodiments, a method is provided for providing drive-through healthcare services to an individual, the method including receiving a request for healthcare services from an individual over a network, the healthcare services to be provided at a drive-through healthcare facility. Upon receiving the request, transmitting an acknowledgement to the individual in response to the received request, the acknowledgement including a questionnaire regarding the individual's need for healthcare services. The method the receives responses to the questionnaire over the network; and, based on the received responses, automatically scheduling a healthcare appointment for the individual to receive the requested healthcare services. Appointment information is transmitted to the individual, the appointment information including a time and a location for the individual to receive the requested healthcare services. The actual provision of healthcare services to the individual is triggered by receiving notice that the individual has arrived at the drive-through facility for the healthcare appointment. Upon arrival of the individual at the drive-through facility, healthcare services are provided by a healthcare provider to the individual while the individual remains in a vehicle, the healthcare provider having access to the individual's health information over the network. At the completion of the services, discharging the individual from the drive-through healthcare facility, the discharging step including transmitting to the individual a referral for additional healthcare services if needed; and transmitting to the individual a summary of healthcare services provided, including any recommended additional healthcare services and any recommended prescriptions.

Under a further embodiment, a method is provided for providing drive-through healthcare services to an individual, the method including receiving notice that an individual has arrived at the drive-through healthcare facility without an appointment, the notice including information whereby the individual can be contacted over a phone, computer, or network. Upon sensing a patient's arrival, transmitting a drive-up questionnaire to the individual, the questionnaire including questions regarding why the individual is seeking healthcare services. Upon receiving responses to the drive-up questionnaire, automatically analyzing the received responses to determine whether appropriate healthcare services are available to assist the individual. If the appropriate healthcare services are available, automatically transmit an acknowledgement to the individual to proceed through the drive-through healthcare facility to receive the healthcare services. If the appropriate healthcare services are not available, automatically transmit to the individual one of a proposed appointment time and drive-through healthcare facility location information; or a referral to receive healthcare services at a non-drive-through healthcare facility.

According to additional embodiments, a system is provided for facilitating drive-through healthcare services to an individual, the system including a communications network transmitting information between an individual and a healthcare provider; a processing device; and computer readable data storage media storing instructions that, when executed by the processing device, cause the processing device to provide a drive-through healthcare service system to the individual. The drive-through healthcare services system is operative to receive a request for healthcare services from an individual over a network, the healthcare services to be provided at a drive-through healthcare facility. The system further transmits an acknowledgement to the individual in response to the received request, the acknowledgement including a questionnaire regarding the individual's need for healthcare services. The system receives responses to the questionnaire over the network; and, based on the received responses, schedules a healthcare appointment for the individual to receive the requested healthcare services. The system transmits the appointment information to the individual; and receives notice from the individual of acceptance of the appointment. The system receives notice that the individual has arrived at the drive-through facility for the healthcare appointment. Healthcare services by a healthcare provider are provided to the individual while the individual remains in a vehicle, the healthcare provider having access to the individual's health information over the network. The system provides for the discharge the individual from the drive-through healthcare facility.

An alternate embodiment provides a computer readable storage device including executable instructions for providing healthcare services to an individual, which when executed by a processor, is operable to receive a request for healthcare services from an individual over a network, the healthcare services to be provided at a drive-through healthcare facility. Upon receiving the request, the instructions provide for transmitting an acknowledgement to the individual in response to the received request, the acknowledgement including a questionnaire regarding the individual's need for healthcare services. A response to the questionnaire is received over the network. Based on the received responses, the instructions provide for scheduling a healthcare appointment for the individual to receive the requested healthcare services. The appointment information is transmitted to the individual; and an acknowledgement of acceptance of the appointment is received from the individual. Notice is received that the individual has arrived at the drive-through facility for the healthcare appointment; and healthcare services are provided to the individual by a healthcare provider while the individual remains in a vehicle, the healthcare provider having access to the individual's health information over the network. The individual is discharged from the drive-through healthcare facility, the discharging step including transmitting to the individual a referral for additional healthcare services if needed; and transmitting to the individual a summary of healthcare services provided, including any recommended additional healthcare services and any recommended prescriptions.

The drive-through healthcare provision system permits individuals (i.e., patients) to conveniently receive healthcare services relatively local to the person's location while remaining in their vehicle. Consistent with the concept of providing one-stop medical services, the providers can know in advance the individual's concerns and symptoms, have access to the individual's medical records, and have assembled personnel and resources to address the patient's individual needs.

The system provides significant improvements over contemporary access to medical services, where a patient must travel to an often distant, centralized medical facility for diagnosis and help and must mingle with likely ill and contagious patients while seeking their own medical assistance. The system provides a simple, convenient, and reliable way for a patient, particularly an individual under stress and in discomfort, to connect with healthcare resources assembled to meet her particular situation. Time is not lost on multiple, sequential appointments because all anticipated resources have been queued to diagnose and treat the patient. Medicines can be prescribed and provided during the same visit, thereby further reducing the time needed for the patient to start receiving remedial healthcare services.

The systems and methods described herein further provide a technological improvement in that healthcare providers not only have access to the individual's healthcare record from the time of making an appointment through the entire diagnosis and treatment process, but can also have access to that information while speaking with the individual while she remains conveniently secure in her vehicle. Access to this information and updates to this information can be made in real time by the healthcare provider. Further, updates, diagnoses, treatment summaries, prescriptions, and subsequent appointments can be transmitted to the individual in real time and near real time so the healthcare provider and the individual are dealing with the same information regarding the individual's health and treatments.

The methods and apparatuses of the present invention have other features and advantages which will be apparent from or are set forth in more detail in the accompanying drawings, which are incorporated herein, and the following Detailed Description, which together serve to explain certain principles of the present invention.

### BRIEF DESCRIPTION OF THE DRAWINGS

The present invention has other features and advantages which will be apparent from or are set forth in more detail in the accompanying drawings, which are incorporated herein, and the following Detailed Description, which together serve to explain certain principles of the present invention and to enable a person of ordinary skill in the art to make and use the embodiments disclosed herein. In the drawings, like reference numbers indicate identical or functionally similar elements.

FIG. 1 illustrates an example architecture of a network for implementing a system and method for providing drive-through healthcare services.

FIG. 2 is a flowchart illustrating the steps of one embodiment for providing drive-through healthcare services.

FIG. 3 is a flowchart illustrating the steps of an alternate embodiment for providing drive-through healthcare services.

FIG. 4 is a flowchart illustrating the steps of yet another embodiment for providing drive-through healthcare services.

It should be understood that the appended drawings are not necessarily to scale, presenting a somewhat simplified representation of various features illustrative of the basic principles of the invention. The specific design features of the present invention as disclosed herein, including, for example, specific dimensions, orientations, locations, and shapes can be determined in part by persons of ordinary skill in the art for the particular intended application and use environments.

### DETAILED DESCRIPTION

In recent years, many drive-through facilities have been developed for providing products and services at the convenience of consumers arriving at a business for receiving products. Drive-through fast food restaurants, drive-through windows at banks, and drive-through windows at pharmacies are a few such facilities. In each such facility, the consumer drives through a dedicated lane to a window for receiving goods. The consumer either drives a vehicle to the facility or is a passenger in the vehicle, and remains in the vehicle for receiving the product. Payment is typically made prior to receiving the products. More recently, under the crisis of the coronavirus, drive-through facilities were created to provide for diagnostic-only purposes to determine whether the individual had contracted the virus. Other drive-through facilities were developed for administering the coronavirus vaccine shot. Each of these drive-through facilities are single-purpose facilities to provide a single product or a single service. None of them are connected to a network for receiving individual healthcare information, and none of them are provisioned to receive a request for healthcare services and to assemble a team at a given appointment time to address such individual requests. Nor are they equipped to response to a drive-up patient with one or more healthcare issues who needs focused diagnostic and treatment resources.

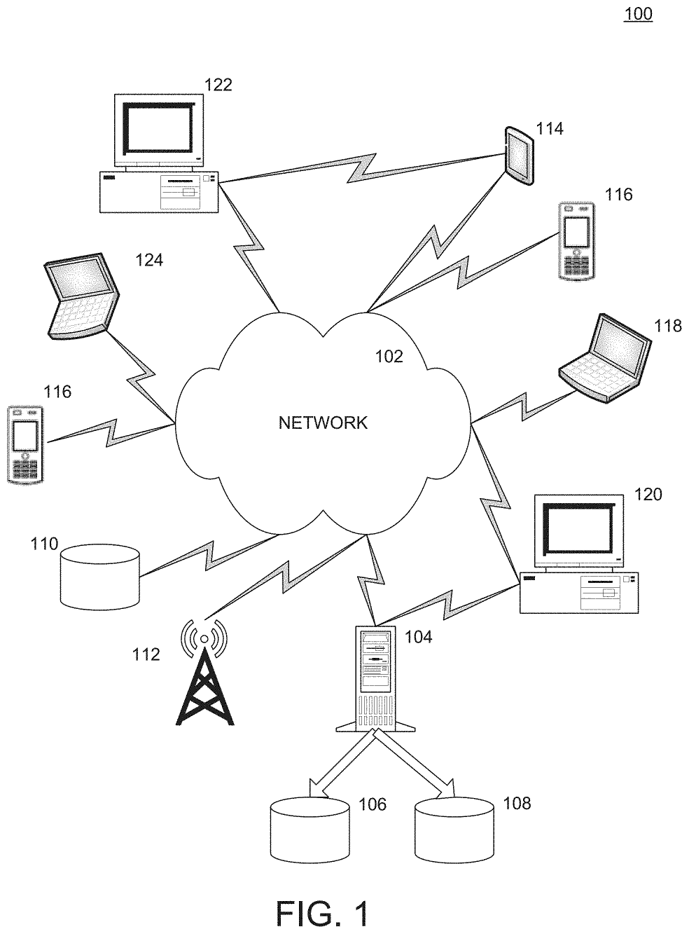

Although the described embodiments can be implemented in any appropriate type of network system supporting any suitable communication standards and using any suitable components, particular embodiments can be implemented in an exemplary network such as shown in FIG. 1.

Referring first to FIG. 1, there is shown an exemplary architecture of a network system 100 for implementing a method for providing drive-through healthcare services to individuals. The network 102 across which communications in exemplary embodiments occur, can include any private or public, wired or wireless network, including but not limited to Local Area Networks, Wide Area Networks, the Internet, the World Wide Web, radio frequency (RF), Bluetooth, and a Cloud-based network. There is shown an exemplary central network server 104 and one or more databases and/or storage devices 106 and 108. These storage devices conveniently store individual medical histories along with medical diagnostic protocols and treatment regiments, all securely accessible by the drive-through healthcare staff. For example, the staff may be required to provide logon information to access the stored information, the stored information may be encrypted, and the stored information may be securely backed up for redundancy. There may be more servers, more databases, and more storage devices 110 than those displayed in FIG. 1, with the servers minimally configured with memory, at least one processor or processing device, communication resources, and with the databases being external to or integrated with the servers. Regional and central network servers will be collectively referred to as the network server 104 or the regional and/or central network servers 104. There are one or more user devices 114-120 for utilizing the drive-through healthcare system and for making healthcare appointments, receiving information regarding the health and treatment of the individual, and for making payment arrangements. There are also one or more healthcare provider devices 124-126 for communicating with the healthcare provider system and patients. Through devices 114-120, devices 124-126, and the network 102, the healthcare providers and the patients can communicate for purposes of accessing, displaying or viewing objects, receiving information, entering information, and communicating information and messages. Patients and patient devices are collectively represented as a smart phone 114. Healthcare providers and provider devices are collectively represented as a tablet 124. The system 100 can effect the access to and the communication of healthcare information for providing diagnosis and treatment information to both the provider and the patient. The drive-through healthcare system 100 extends to software, programs, and routines within storage media on each of the patient devices, provider devices, and servers.

The patient devices 114 for initiating an appointment and for receiving personalized healthcare information are representatively shown as a smart phone 114, a cell phone 116, a portable tablet or laptop computer 118, and a desktop computer 120. While not completely shown in FIG. 1, healthcare provider devices include similar types of equipment. Examples of patient devices 114 and provider devices 124 include, but are not limited to, wireless user equipment and communication devices, such as, for example, mobile telephones, smart phones, personal digital assistants, electronic readers, portable electronic tablets, personal computers, and laptop computers. Each representative patient device 114 and provider device 124 minimally comprises a processor or processing device, a memory coupled to the processor, a display coupled to the processor, computer readable media, facilities for entering information into the patient device 114 or provider device 124, and an antenna or other wired or wireless connection device coupled to the processor for receiving and transmitting information, messages, and/or commands or instructions. The display can include touch screen technology for the entry of patient, treatment, diagnosis, and payment information provided and/or required by the drive-through healthcare system. The information can be entered, for example and not limitation, in text form or by touching action buttons displayed on the screen or integrated with the body of the device 114/124. Alternately, patient entry of information and reactions can be through use of a physical or touch screen keyboard or by voice.

Portions of the system are envisioned to be encoded into a drive-through healthcare application loaded onto a patient's device 114 for access by the patient as a need may arise. Each patient can download the application from an app store if the application is not already preloaded on the patient's device 114. Under such circumstances, standard or default information can be set up within each such application, providing for a list of nearby drive-through healthcare facilities available to provide medical services.

While FIG. 1 shows all communications being directed to the network server 104, exemplary embodiments are not so limited; and communications can be provided directly through the network 102 for the patient devices 114 and the providers 124. Alternately, communications can be provided directly between a remote patient device and one or more providers as shown between patient device 114 and provider device 124.

Exemplary embodiments are implemented on the computers of the patient devices 114 and the provider devices 124 and, alternately the network server 104. Computer executable instructions, or software, are provided for directing the processing of the computers of the exemplary devices 114, 124, and 104, including processing the steps of exemplary embodiments of the alert messaging system 100. The computer executable instructions, when executed by the computers 114, 124, and 104 and/or the processors associated with each of said computers, provide for the exchange of information and the performance of the steps of providing healthcare services to individuals.

One or more storage devices 106, 108, and 110 are provided for storage of and access to information regarding the individual's medical records, diagnostic aids and resources, treatment options, and regional healthcare providers for the delivery of healthcare services to individuals. The system is configured to secure this information for privacy concerns as discussed more thoroughly below.

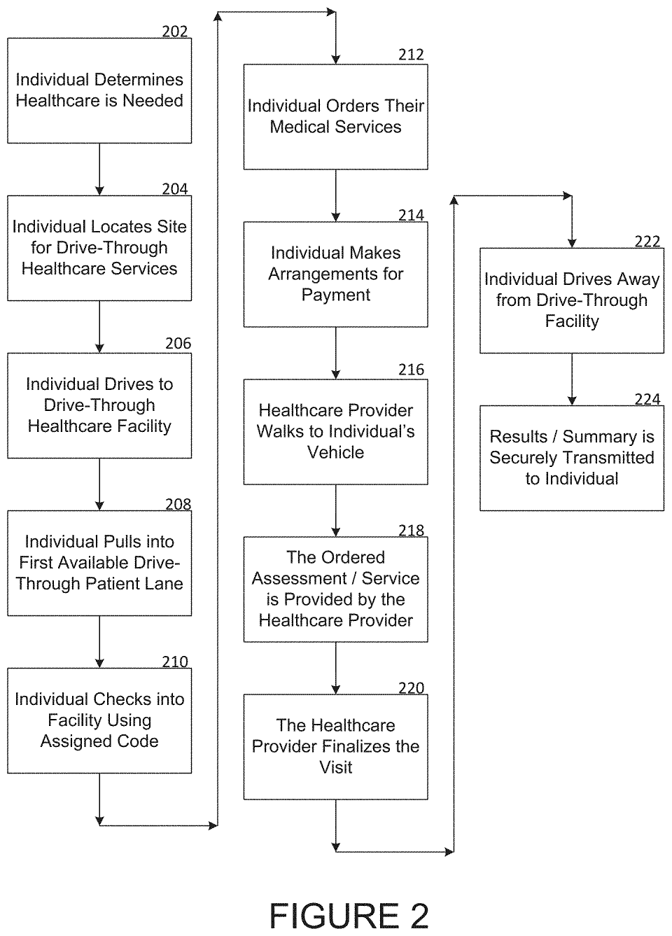

Computer readable media includes computer storage media, which includes volatile and non-volatile media, removable and non-removable media implemented in any method or technology for the storage of information, including computer readable instructions, data structures, display templates, and provider information. Computer storage media includes, but is not limited to magnetic media (e.g., a hard disk), non-transitory memory, optical media (e.g., a DVD), memory devices (e.g., random access memory), and the like. In some embodiments, computer readable instructions are configured such that, when executed by a processor and/or a processing device, the instructions cause the exemplary computers 114, 124, and 104 to perform steps described below of the drive-through healthcare provider system (e.g., steps described below with reference to the flow chart shown at least in FIGS. 2, 3, and 4). In other embodiments, the exemplary computers 114, 124, and 104 are configured to perform steps described below without the need for software instructions. Therefore, the features of the present embodiments described herein may be implemented in any suitable combination of hardware and/or software. Computer storage media does not include a carrier wave or any other propagated data signal.

Exemplary embodiments provide an improved method and system for providing healthcare services to individuals with drive-through facilities. The disclosed system provides automated appointments, diagnosis, treatment, and medical reporting to individuals in need of medical services. As such, the system provides significant, potentially life-saving benefits over existing medical treatment systems by, for example, avoiding busy centralized medical treatment centers where the patients could be additionally infected, avoiding time-consuming serial medical treatments across multiple appointments and locations, and receiving diagnosis, treatment, and medical summaries all in one stop, from the convenience of the patient's vehicle. As drive-through restaurants and banks have recently closed or abandoned their customer facilities, these locations are now becoming available for the drive-through healthcare facilities described herein. As such, the cost of providing such drive-through healthcare services is significantly less expensive than medical services provided at a centralized medical facility and its requisite large building, staff, and equipment.

Referring now to FIG. 2, there is shown a flowchart of a method for providing drive-through healthcare services in accordance with exemplary embodiments of the present invention. The present invention provides a method for providing healthcare diagnostic and treatment services at a drive-through healthcare facility. Hereinafter, the steps of this method will be described in detail.

At step 202, an individual determines that she is in need of healthcare services. This may be an individual choice, a choice prompted by another person acquainted with the individual, or may be time for a routine checkup. The individual (alternately, patient or user) accesses the drive-through healthcare services web site or portal over the network 102 by means of the individual's device 114. Alternately, the individual can access the drive-through healthcare services provider through a drive-through application on the individual's device 114. At step 204, the individual determines, from the web site, portal, or the application, a location for drive-through healthcare services that is convenient to the individual. The web site, portal, or the application can identify particular medical services that are available at each drive-through healthcare facility, thereby permitting the individual to select a facility that may be best equipped to address her medical concerns.

The individual may have the option of notifying the particular facility that she is coming and receive a patient code identifying herself. She also has the option of notifying the particular facility of the services she would like to receive or the medical issues she is facing, so the facility may be able to better prepare for her arrival, diagnosis, and treatment. She then drives to that facility at step 206. The drive-through facility may be a repurposed fast food restaurant or bank having at least one drive-through lane. Alternately, the drive-through facility could be a purpose built healthcare facility for providing drive-through healthcare services. Upon arriving at the drive-through facility, the individual pulls into the first available incoming patient lane at step 208. In this manner, medical services are typically provided on a first-come, first-served basis. The individual's arrival at the facility is detected, and the individual is greeted and asked why she has come to the facility. Detection of the individual's arrival can be accomplished through several means, some automated and some human-monitored. For example, the arrival of the individual's vehicle can automatically be sensed by detection sensors 112 in the lane pavement or by infrared or optical sensors 112 that detect the vehicle. Alternately, staff at the facility can monitor a display of the incoming lanes and notice the individual's arrival. As the vehicle approaches the facility in an incoming lane, a staff member who has been notified of the individual's arrival, greets and checks the individual into the facility at step 210, either in person by walking to the vehicle or through a speaker adjacent to the incoming lane. Alternately, the individual can check in by using the drive-through application on their device 114, optionally with the received patient code.

As part of the check-in process, the individual identifies herself, either with logon information if she is already a client of the healthcare provider or else with profile information if she is a new subscriber or patient to the provider services. Such profile information can include name, address, date of birth, gender, health insurance information and links to the health insurance provider, demographic information, and preferred appointment times and locations.

At step 212, the individual orders their medical services. This step can take the form of the individual explaining to the staff member, through the speaker, or through the drive-through application on the individual's device 114 why she has come to the drive-through facility. The individual can explain she has arrived for a routine checkup or can explain her medical symptoms and concerns. Alternately, the individual can select services from a menu displayed on the individual's device 114 or on a display adjacent to the vehicle. This information is entered into the facility's drive-through system, and the system automatically determines the services to be provided, who on staff at the facility will provide the services, whether additional information is needed, and the cost of the services. If additional information is needed, a questionnaire is automatically generated by the system, based on the individual's information provided so far, and transmitted to the individual through the drive-through application of her device 114 or, alternately through a staff member at the vehicle or a speaker at the vehicle. The individual responds to the questionnaire, and the answers are received by the healthcare system, which automatically determines a course of treatment based on the information provided by the individual.

The system automatically analyzes the resources required by the determined course of treatment and compares those needed resources against the resources available at that drive-through healthcare facility. If the needed resources are available, the system transmits an acceptance and acknowledgement to the individual to proceed through the healthcare facility to receive the determined healthcare services. If the appropriate healthcare services are not available, the system automatically transmits to the individual one of a proposed appointment time and drive-through healthcare facility location information, or a referral to receive healthcare services at a non-drive-through healthcare facility. If the system determines the individual's condition requires urgent care, the system will so notify the individual and the healthcare staff and provide for an expedited appointment at the nearest facility that can address the individual's needs. These urgent steps can include the dispatching of an ambulance to the drive-through healthcare facility's location.

The drive-through healthcare system and facilities are networked with other medical services providers, ranging from large, centralized facilities to medical specialists to small medical clinics. Through such networking and resource information available through the drive-through healthcare system, the system can refer patients to other, appropriate medical facilities when such a referral is in the patient's best interests or when the drive-through facility lacks the resources necessary to treat the patient, for example. Not all drive-through facilities provide the same resources or level of care as others, so a referral system expands the services that can be provided through the drive-through healthcare system by tapping into the medical resources available at other drive-through healthcare facilities and at non-drive-through healthcare facilities.

If the needed resources are available, treatment of the individual proceeds with the projected cost of the treatment being transmitted to the individual, who makes arrangements for payment at step 214. The treatment cost transmitted to the individual can include a breakdown of costs, including the services to be provided and how much of the cost the individual's health insurance is anticipated to cover. This cost information can also include alternate forms of payment that may be available to cover the projected cost. The payment arrangements can include providing a credit card for payment and/or providing additional health insurance information for subsequent submission of a claim by the healthcare provider.

At step 216, the appropriate healthcare provider designated by the system approaches the vehicle, often bringing the necessary diagnostic and treatment devices for providing the needed treatment for the individual, along with a provider device 124 for accessing medical information including diagnostic information, symptom information, and the individual's medical history. At step 218, the provider provides the requested/ordered/needed assessment and/or service to the individual. During this step of providing the medical services, the provider can provide a live feed through her device 124 to authorized, interested medical personnel, including staff within the drive-through facility, the individual's primary care provider, and medical experts relevant to the individual's diagnosis and treatment. Similarly, data from tests and treatments being performed on the individual can be streamed and/or transmitted for viewing, analysis, and secure storage. At step 220, the provider finalizes the visit. During these last two steps, the provider administers the needed diagnostic services and treatment services requested by the individual and/or called for by the individual's medical condition. The provider can utilize her device 124 to access needed medical information to determine the needed treatment, to request additional providers from the facility to assist her, to update the individual's medical history, to order prescriptions, and to order subsequent follow-up at the current facility or at another medical facility.

At step 222, the individual drives away from the drive-through medical facility, having completed the requested/needed medical services. The individual may have received a prescription at the facility as part of her treatment. At step 224, a summary and results of her medical diagnosis and/or treatment is securely transmitted to the individual, who can immediately access the information on her device 114 upon providing proper credentials. The summary can include information regarding a prescription that has been transmitted to a pharmacy of her choice, additional medical appointments that have been made for her, and any follow-up appointments to be made for her continued medical care. The individual's medical history is correspondingly updated with the diagnosis and treatments that were provided, along with any medicines or future visits that were prescribed. With the individual's consent, the diagnosis and treatment information can also be transmitted to the individual's primary care provider. Subsequent test results and follow up contacts can be made from the drive-through healthcare system to the individual's device 114 by transmitting the results and inquiries from the drive-through healthcare system. The system can thereby follow up with the individual's continuing medical care.

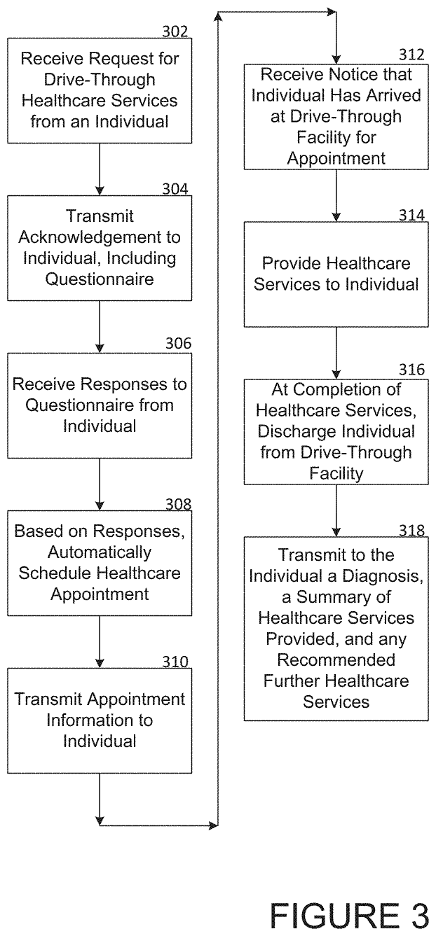

Referring now to FIG. 3, there is shown a flowchart of an alternate method for providing drive-through healthcare services in accordance with exemplary embodiments of the present invention. In this embodiment, instead of driving to the drive-through healthcare facility without a prior appointment, this embodiment provides for the individual to make an advance appointment to utilize the facilities of the drive-through facility. In this embodiment, an individual determines that she is in need of healthcare services, as discussed above. The individual accesses the drive-through healthcare services web site, portal, or web site as also discussed above. The individual identifies herself, either with logon information if she is already a client of the provider or else with profile information if she is a new subscriber or patient to the provider services. Such profile information can include name, address, date of birth, gender, health insurance information and links to the health insurance provider, demographic information, and preferred appointment times and locations. The individual also provides a reason for the healthcare visit and/or a description of her medical issue, such as a routine health checkup, an aching joint, a critical injury, symptoms that are of concern, and the like. The individual has the option of selecting a particular drive-through facility from the information on the healthcare services' web site, portal, or application. All of this individual-provided information is transmitted or conveyed to the healthcare system, which receives the information as a request for medical services to be provided at a drive-through healthcare facility.

The healthcare system, upon receipt of the inquiry from the individual at step 302, automatically reviews and analyzes the received information to determine the nature of the request for medical services and determines what additional information is needed to properly schedule an appointment at an adequately equipped and staffed drive-through healthcare facility for the individual. Included in this analysis is an identification of possible healthcare concerns for the individual. At step 304, the healthcare system automatically transmits an acknowledgement to the individual, providing notice that the system is aware of her needs and is preparing for her visit. The acknowledgement includes a questionnaire that has been automatically customized and composed by the healthcare system, requesting additional information based on the individual's description of the services she is seeking, the symptoms of her condition, or the ailments she is experiencing. At step 306, the system receives the answers to the questionnaire from the individual over the network and/or conveyed through the drive-through application, the answers being analyzed by the system to determine the staff and equipment (i.e., resources) needed to address the healthcare issues of the individual and subsequently to search for and determine those drive-through healthcare facilities that are available and equipped to address the medical issues of the individual. Date and time priority is given to individuals having urgent medical needs. Under a scenario of the individual having serious or urgent medical issues, priority can be provided by directing the individual to the nearest facility that can address her needs and a code that identifies her urgency upon arrival at the facility so prompt attention and treatment can be provided. Location priority is given to those sufficiently equipped facilities nearest to the location of the individual. The determination of sufficiently equipped facilities includes a review of available equipment and staff.

Based on the information received from the individual and the available healthcare resources, the system automatically makes an appointment for the individual to visit a particular drive-through healthcare facility and transmits appointment information to the individual at step 310. The appointment information includes the time and date of the appointment as well as the location of the designated facility. The system also transmits the appointment information to the designated facility, allocating the equipment and personnel resources at that facility for a given period of time for the appointment. In this manner, the system maintains an inventory of available resources for continued scheduling of resources and patients so that the resources are efficiently utilized and overbookings are avoided. As a form of redundancy, at a predetermined time, such as two hours, prior to an appointment, the system transmits a confirmation of the appointment to the designated drive-through healthcare facility, requesting acknowledgement that the specific needed healthcare staff and equipment will be available for the individual at the time of the appointment.

The transmitted appointment information can also include a code unique to the individual for identifying herself upon arrival at the facility, an estimated cost for the services to be provided, and a request for acceptance of the appointment. The individual can respond by transmitting acceptance of the appointment.

At step 312, the designated drive-through facility receives notice or a signal that the individual has arrived for her appointment, as discussed in more detail above. Since the drive-through healthcare system has made the appointment, the individual's medical history and medical issues are readily available to the facility online by the time the individual arrives, as are the needed medical resources. At step 314, the scheduled healthcare services are provided to the individual while she waits in her vehicle. At step 316, at the completion of the scheduled healthcare services, the individual is discharged from the facility. At step 318, the system transmits to the individual a summary of her diagnosis and treatment, the services provided, and any recommended further healthcare services and referrals. This information is also used to update the individual's medical history.

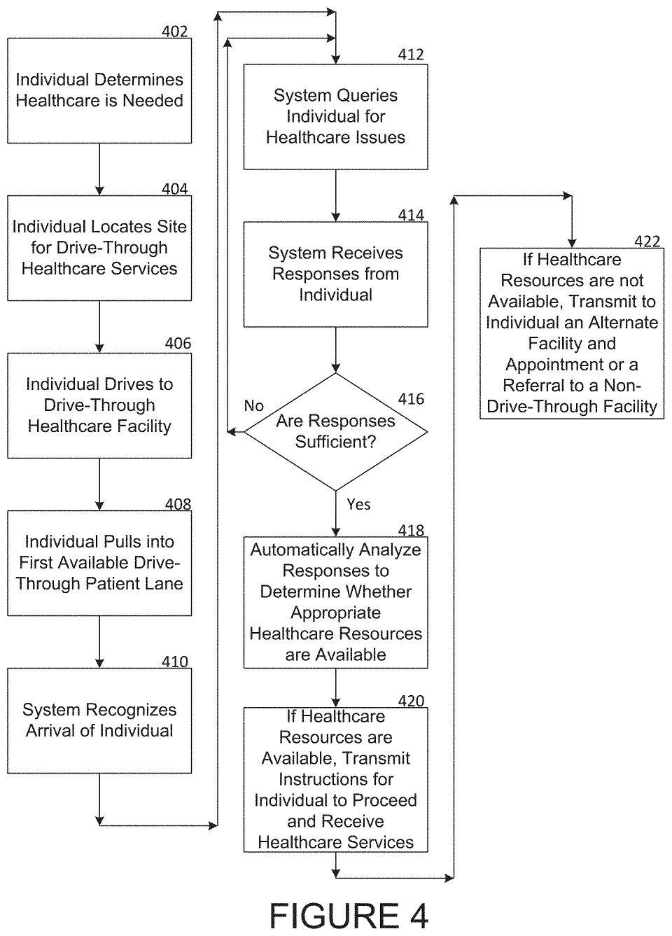

Referring now to FIG. 4, there is shown a flowchart of a yet another method for providing drive-through healthcare services in accordance with exemplary embodiments of the present invention. In this embodiment, additional options are presented to the individual who elects to drive to a drive-through healthcare facility without a prior appointment. In this embodiment, steps 402-408 are similar to steps 202-208 shown in FIG. 2 and discussed above. At step 410, the system recognizes that a vehicle has entered a drive through lane of the facility. At step 412, the system queries the individual for the healthcare issues that prompted her to drive to the facility. As discussed above, these queries can be made to the individual through a speaker located at the drive through lane, through a healthcare staff member who walks to the vehicle, through the drive-through application on the individual's device 114, or through a medical services menu displayed on the individual's device 114 or on a display adjacent to the vehicle.

At steps 414 and 416, the healthcare system receives the responses as entered or transmitted by the individual and determines whether the responses provide a sufficient explanation of the issues being experienced by the individual. If the responses are insufficient, the system sends or transmits follow-up questions to the individual to answer and send back to the system. In other words, if the system cannot make a definitive diagnosis and/or determine a specific treatment plan, the system will generate and transmit additional questions to the individual. For example, the individual may have been vague regarding what type of checkup is requested, or the individual has not provided enough specific information regarding the circumstances under which her knee hurts.

At step 418, which overlaps with the analysis performed at step 416, the system determines the services and treatment needed to address the individual's healthcare issues and whether appropriate resources in the form of healthcare staff and equipment are available at that particular location to treat the individual. At step 420, if the needed healthcare resources are available, instructions are transmitted to the individual and the appropriate healthcare staff to proceed with providing the healthcare services.

At step 422, if the healthcare resources are not available at that particular drive-through healthcare facility, the system determines where such services would be available, again with a priority for recommending facilities that are closer to the individual's preferred location. The system determines when times are available at several of the closest facilities to address the individual's issues and transmits those times and locations to the individual. The individual can then respond, accepting one of the appointments, acceptance of which is automatically transmitted to the selected drive-through healthcare facility to reserve the healthcare resources at that appointed time and location. Alternately, the system can refer the individual to a non-drive-through healthcare facility for receiving treatment.

The present drive-through healthcare system is configured to analyze an individual's medical concerns, issues, and symptoms; and automatically diagnose the individual's medical issues and develop a treatment plan to address those issues—all within a single visit to the drive-through healthcare facility. Medical staff at the drive-through healthcare system can efficiently supplement the diagnosis and treatment steps by working directly with the individual while the individual remains within the convenience and comfort of her vehicle. By utilizing such automated features as vehicle detection at the facility, determining initial questions to pose to the individual based on the individual's issue identification, determining what additional information is needed from the individual, determining whether resources are available at the facility to provide the recommended treatment, and creating appointments at other drive-through and non-drive-through healthcare facilities, the drive-through healthcare system provides significant technological improvements over contemporary healthcare providers. Individuals can receive complete, one-stop treatment without having to leave their vehicles and can do so with minimal risk of being exposed to sick persons at the healthcare facility. Costs to the individual and time expended can be minimized by a single healthcare facility visit where all needed resources are assembled to provide recommended treatment to the individual.

The foregoing descriptions of specific exemplary embodiments of the present invention have been presented for purposes of illustration and description. They are not intended to be exhaustive nor to limit the invention to the precise forms disclosed, and obviously many modifications and variations are possible in light of the above teachings. The exemplary embodiments were chosen and described in order to explain certain principles of the invention and their practical application, to thereby enable others skilled in the art to make and utilize various exemplary embodiments of the present invention, as well as various alternatives and modifications thereof. It is intended that the scope of the invention be defined by the Claims appended hereto and their equivalents.

What is claimed is:

1. A method for providing drive-through healthcare services to an individual, comprising:
receiving a request for healthcare services from an individual over a network, the healthcare services to be provided at a drive-through healthcare facility;
transmitting an acknowledgement to the individual in response to the received request, the acknowledgement including a questionnaire regarding the individual's need for healthcare services;
receiving responses to the questionnaire over the network;
based on the received responses, automatically scheduling a healthcare appointment for the individual to receive the requested healthcare services;
transmitting appointment information to the individual, the appointment information comprising a time and a location for the individual to receive the requested healthcare services;
receiving notice that the individual has arrived at the drive-through facility for the healthcare appointment;
providing healthcare services by a healthcare provider to the individual while the individual remains in a vehicle, the healthcare provider having access to the individual's health information over the network; and
discharging the individual from the drive-through healthcare facility, the discharging step comprising:
transmit to the individual a referral for additional healthcare services if needed; and
transmitting to the individual a summary of healthcare services provided, including any recommended additional healthcare services and any recommended prescriptions.

2. The method of claim 1, further comprising displaying an options screen to an individual for requesting healthcare services, the screen display including fields to enter individual information comprising one or more of:
login information;
personal individual information;
description of healthcare concern;
description of healthcare symptoms;
preferred appointment times and locations; and
links to the individual's healthcare information.

3. The method of claim 1, further comprising the automated steps of:
reviewing the received healthcare services request;
identifying possible healthcare concerns for the individual;
determining whether more information is needed regarding the identified possible healthcare concerns; and
composing questions for the questionnaire to obtain the needed information.

4. The method of claim 3, further comprising automatically determining healthcare services to be provided to the individual based on the received healthcare request and the received responses to the questionnaire.

5. The method of claim 4, further comprising searching for at least one drive-through facility available to the individual based on location of the individual and the determined healthcare services to be provided.

6. The method of claim 1, further comprising the received responses including a link to the individual's healthcare information.

7. The method of claim 1, wherein the transmitted appointment information further comprises:
a location of the drive-through facility;
a code unique to the individual and the appointment;
an estimated cost of the requested/scheduled healthcare services; and
a request for acceptance of the appointment.

8. The method of claim 1, further comprising receiving an acceptance of the appointment from the individual.

9. The method of claim 1, further comprising allocating healthcare resources to be available at the time and location of the appointment based on the requested healthcare services and the received responses.

10. The method of claim 1, further comprising, at a predetermined time prior to the scheduled appointment, verifying that the allocated healthcare resources for the appointment will be available at the appointment time and location.

11. A method for providing drive-through healthcare services to an individual, comprising:
receiving notice that an individual has arrived at the drive-through healthcare facility without an appointment, the notice including information whereby the individual can be contacted over a phone, computer, or network;
transmitting a drive-up questionnaire to the individual, the questionnaire including questions regarding why the individual is seeking healthcare services;
receiving responses to the drive-up questionnaire;
automatically analyzing the received responses to determine whether appropriate healthcare services are available to assist the individual;
if the appropriate healthcare services are available:
automatically transmitting an acknowledgement to the individual to proceed through the drive-through healthcare facility to receive the healthcare services; and
providing the appropriate healthcare services to the individual;
if the appropriate healthcare services are not available, automatically transmitting to the individual one of:
a proposed appointment time and drive-through healthcare facility location information; or
a referral to receive healthcare services at a non-drive-through healthcare facility.

12. A system for providing drive-through healthcare services to an individual, the system comprising:
a communications network transmitting information between an individual and a healthcare provider;
a processing device; and
computer readable data storage media storing instructions that, when executed by the processing device, cause the processing device to provide a drive-through healthcare service system to the individual, the drive-through healthcare services system operative to:
receive a request for healthcare services from an individual over a network, the healthcare services to be provided at a drive-through healthcare facility;
transmit an acknowledgement to the individual in response to the received request, the acknowledgement including a questionnaire regarding the individual's need for healthcare services;
receive responses to the questionnaire over the network;
based on the received responses, schedule a healthcare appointment for the individual to receive the requested healthcare services;
transmit the appointment information to the individual;
receive notice from the individual of acceptance of the appointment;
receive notice that the individual has arrived at the drive-through facility for the healthcare appointment;
provide healthcare services by a healthcare provider to the individual while the individual remains in a vehicle, the healthcare provider having access to the individual's health information over the network; and
discharge the individual from the drive-through healthcare facility.

13. The system of claim 12, wherein the drive-through healthcare service system is further operative to:
review the received healthcare services request;
identify possible healthcare concerns for the individual;
determine whether more information is needed regarding the identified possible healthcare concerns; and
compose questions for the questionnaire to obtain the needed information.

14. The system of claim 13, wherein the drive-through healthcare service system is further operative to automatically determine healthcare services to be provided to the individual based on the received healthcare request and the received responses to the questionnaire.

15. The method of claim 14 wherein the drive-through healthcare service system is further operative to search for at least one drive-through facility available to the individual based on location of the individual and the determined healthcare services to be provided.

16. The method of claim 12, wherein the transmitted appointment information further comprises:
a date and time of the appointment;
a location of the drive-through facility;
a code unique to the individual and the appointment;
an indication whether a support individual is required;
an estimated cost of the requested/scheduled healthcare services; and
information whereby the cost of the requested healthcare services can be paid.

17. The method of claim 12, wherein the drive-through healthcare service system is further operative to allocate healthcare resources to be available at the time and location of the appointment based on the requested healthcare services and the received responses.

18. A computer readable storage device including executable instructions for providing healthcare services to an individual, which when executed by a processor, is operable to:
receive a request for healthcare services from an individual over a network, the healthcare services to be provided at a drive-through healthcare facility;
transmit an acknowledgement to the individual in response to the received request, the acknowledgement including a questionnaire regarding the individual's need for healthcare services;
receive a response to the questionnaire over the network;
based on the received responses, schedule a healthcare appointment for the individual to receive the requested healthcare services;
transmit the appointment information to the individual;
receive acknowledgement of acceptance of the appointment from the individual;
receive notice that the individual has arrived at the drive-through facility for the healthcare appointment;
provide healthcare services to the individual by a healthcare provider while the individual remains in a vehicle, the healthcare provider having access to the individual's health information over the network; and
discharge the individual from the drive-through healthcare facility, the discharging step comprising:
transmit to the individual a referral for additional healthcare services if needed; and
transmit to the individual a summary of healthcare services provided, including any recommended additional healthcare services and any recommended prescriptions.

19. The system of claim 18, wherein the device is further operable to:
review the received healthcare services request;
identify possible healthcare concerns for the individual;
determine whether more information is needed regarding the identified possible healthcare concerns; and
compose questions for the questionnaire to obtain the needed information.

20. The system of claim 19, wherein the device is further operable to automatically determine healthcare services to be provided to the individual based on the received healthcare request and the received responses to the questionnaire.
